(19) United States
(12) Patent Application Publication
(10) Pub. No.: US 2024/0131034 A1
(43) Pub. Date: Apr. 25, 2024

(54) METHODS OF TREATING STAPHYLOCOCCUS AUREUS BACTEREMIA INFECTIONS

(71) Applicant: Basilea Pharmaceutica International AG, Allschwil, Allschwil (CH)

(72) Inventors: Marc ENGELHARDT, Allschwil (CH); Mark Edward Jones, Allschwil (CH)

(21) Appl. No.: 18/483,703

(22) Filed: Oct. 10, 2023

Related U.S. Application Data
(60) Provisional application No. 63/414,635, filed on Oct. 10, 2022.

(51) Int. Cl. A61K 31/546; A61P 31/04

(52) U.S. Cl. CPC A61K 31/546; A61P 31/04

(57) ABSTRACT

The present invention provides methods of treating a S. aureus bloodstream infection (bacteremia) to a patient in need thereof, the method comprising administering ceftobiprole as ceftobiprole miedocaril to the patient at a dosage corresponding to 500 mg of ceffobiprole every 6 hours on days 1 to 8 and 500 mg every 8 hours on days 9 onwards.

### STATEMENT REGARDING FEDERALLY SPONSORED RESEARCH OR DEVELOPMENT

This invention was made with United States Government support under contract number HHSO100201600002C awarded by the Biomedical Advanced Research and Development Authority (BARDA). The United States Government has certain rights in the invention.

### FIELD OF THE INVENTION

The present invention relates to methods of treating Staphylococcus aureus bacteremia infections using ceftobiprole, administered as the prodrug ceffobiprole medocaril. All documents relied upon or cited to below are expressly incorporated herein by reference.

### BACKGROUND OF THE INVENTION

Staphylococcus aureus is a common cause of bacteremia and the leading cause of infective endocarditis in the industrialized world. Recent reviews have suggested an incidence of S. aureus bacteremia (SAB) and infective endocarditis of 16-41 and 3.5-16.6, respectively, per 100,000 person-years. S. aureus accounts for 15-40% of infective endocarditis cases and this proportion is increasing. In adults, 30-day mortality from SAB is estimated at 15-25%, and in-hospital mortality for all causes of infective endocarditis attributable to S. aureus is 22-66% (Tong et al.; Asgeirsson et al.). Moreover, methicillin-resistant S. aureus (MRSA) bacteremia is associated with a significantly higher mortality rate than methicillin-susceptible S. aureus (MSSA) bacteremia, with a recent large, observational cohort study reporting mortality rates of 26 versus 13% (MRSA vs MSSA) in 2013-2015 (Bassetti et al.; Austin et al.; Van Hal et al.). Despite the considerable health burden associated with SAB, there are limited antibiotic treatment options available, particularly for patients with MRSA bacteremia. Furthermore, there remains a lack of robust, high quality evidence supporting the selection of antimicrobial agents, with few randomized, controlled studies in this condition (Thwaites et al.; Holland et al.). Reports of an increasing prevalence of MRSA isolates with reduced susceptibility and poorer responses to agents such as vancomycin and daptomycin (Fowler et al.; Sharma et al.; Gasch et al.; Zhang et al.) further highlight the need for new antibacterial options for SAB (Hamed et al.).

Cettobiprole, the active moiety of the prodrug ceftobiprole medocaril, is an advanced-generation, broad-spectrum cephalosporin γ-lactam agent that has a rapid bactericidal effect against both MSSA and MRSA infections (Líapikou et al.; Morosini et al.) and activity covering isolates of streptococci (including penicillin-resistant pneumococci), Enterococcus faecalis, Haemophilus influenzae, Moraxella catarrhalis, non-extended-spectrum β-lactamase-producing Enterobacteriaceae, and susceptible Pseudomonas aeruginosa (Giacobbe et al.; Pfaller et al.). It is currently approved in many European and non-European countries for the treatment of community- and hospital-acquired pneumonia (excluding ventilator-associated pneumonia) (Hamed et al.).

Recently, the TARGET Phase 3 study demonstrated that ceftobiprole is non-inferior to vancomycin and aztreonam in the treatment of acute bacterial skin and soft tissue infections, in terms of early clinical response and investigator-assessed clinical success (NCT03137173; Overcash et al.). Hamed et al. describes the clinical trial protocol “Ceffobiprole versus daptomycin in Staphylococcus aureus bacteremia: a novel protocol for a double-blind, Phase III trial”. A corresponding clinical trial was initiated under clinical trial number NCT03138733.

It has now been found in a phase three clinical trial for the treatment of S. aureus bacteremia (NCT03138733) that ceftobiprole is non-inferior to daptomycin for overall success at 70 days post-randomization in patients with complicated SAB. All-cause mortality and microbiological eradication rates were similar between treatment groups. Both treatments were well tolerated.

### SUMMARY OF THE INVENTION

The present invention provides methods of treating a Staphylococcus aureus bloodstream infection (bacteremia) to a patient in need thereof, the method comprising administering ceffobiprole as ceftobiprole medocaril to the patient at a dosage corresponding to 500 mg of ceftobiprole every 6 hours on days 1 to 8 and 500 mg every 8 hours on days 9 onwards,

In some embodiments ceftobiprole is administered to the patient for a minimum of 21 days.

In some embodiments ceffobiprole is administered to the patient for up to 42 days.

In some embodiments the SAB infection is complicated SAB.

In some embodiments the treatment achieves microbiological eradication of the infection.

In some embodiments the treatment avoids the formation of new S. aureus metastatic infections.

In some embodiments the treatment avoids any new S. aureus complications.

In some embodiments the patient has right-sided infective endocarditis caused by S. aureus.

In some embodiments the patient has definite right-sided infective endocarditis caused by S. aureus.

In some embodiments the patient has definite native-valve right-sided infective endocarditis caused by S. aureus.

In some embodiments the patient has a bone infection caused by S. aureus.

In some embodiments the bone infection is a non-implant associated bone infection caused by S. aureus.

In some embodiments the bone infection is osteomyelitis caused by S. aureus

In some embodiments the patient has a joint infection caused by S. aureus.

In some embodiments the joint infection is a non-implant associated joint infection caused by S. aureus.

In some embodiments the joint infection is septic arthritis caused by S. aureus.

In some embodiments the patient has an intra-abdominal abscess caused by S. aureus.

In some embodiments the patient has a metastatic infection of native tissue caused by S. aureus.

In some embodiments the patient has an acute bacterial skin and skin structure infection (ABSSSI) caused by S. aureus.

In some embodiments the infection is caused by methicillin-susceptible S. aureus.

In some embodiments the infection is caused by methicillin-resistant S. aureus.

In some embodiments the patient is a chronic intermittent dialysis patient.

In some embodiments the patient has previously received potentially effective treatment with a different antibiotic for the infection.

In some embodiments the patient has persistent SAB.

The invention also provides a pharmaceutical product comprising (I) a container containing ceftobiprole as ceftobiprole medocaril and (ii) instructions for using the ceftobiprole as set out above.

Additional aspects and embodiments of the invention are described in more detail below.

### BRIEF DESCRIPTION OF THE DRAWINGS

FIG. 1 shows the primary efficacy outcome of the phase 3 clinical trial.

FIGS. 2A and 2B show the secondary efficacy outcomes of the phase 3 clinical trials.

FIG. 3 shows the subgroup analyses of the primary outcome for various categories of complicated SAB.

The abbreviations in the figures have the following meanings: ABSSSI: acute bacterial skin and skin structure infections, AZT: aztreonam, CE: clinically evaluable (population), DAP: daptomycin, mITT: modified intent-to-treat (population), MRSA: methicillin-resistant S. aureus, MSSA: methicillin-susceptible S. aureus, PTE: post-treatment evaluation (visit) (at 70 days after randomization), RIE: right-sided infective endocarditis, SAB: S. aureus bacteremia. DAP/AZT means daptomycin with or without aztreonam.

The asterisk in the figures indicates between-group difference of ceftobiprole minus daptomycin ±aztreonam, adjusted for actual stratum (dialysis status and prior antibacterial treatment use) using Cochran-Mantel-Haenszel weights method.

### DETAILED DESCRIPTION OF THE INVENTION

Certain terms used herein are described below. Compounds of the present invention are described using standard nomenclature. Unless defined otherwise, all technical and scientific terms used herein have the same meaning as is commonly understood by one of skill in the art to which this invention belongs.

The term “microbiological eradication” as used herein means no growth of S. aureus pathogen(s) from the presence in blood at baseline, secondary to an adequate clinical response (as determined by a physician), based on a negative blood culture while the patient is on active study treatment, which is confirmed by at least one subsequent negative blood culture for S. aureus at least 24 hours after the first negative blood-culture, without any subsequent S. aureus positive blood culture in the period up to 7 days after end of treatment.

The term “complicated SAB” as used herein refer to patients having at least one positive blood culture for S. aureus at baseline, and having least one of the following: the patient is undergoing chronic intermittent hemodialysis or peritoneal dialysis; persistent SAB; ABSSSI; metastatic infection of native tissue (including but not limited to: septic arthritis or bacterial joint infection/empyema; septic or suppurative thrombophlebitis; visceral soft-tissue abscesses; and septic pulmonary emboli/infarction); definite native-valve right sided infective endocarditis; osteomyelitis (including vertebral, stemal, or long-bone osteomyelitis; epidural or cerebral abscess. The term “SAB complications” has the corresponding meaning.

The term “persistent SAB” is used herein means failure of bloodstream clearance as evidenced by positive blood culture after prior treatment for the SAB with an antibiotic other than ceftobiprole (including other than ceflobiprole medocaril).

The term “pharmaceutically acceptable” as used herein refers to items such as compounds, materials, compositions and/or dosage forms, which are, within the scope of sound medical judgment, suitable for contact a human, without excessive toxicity or other complications commensurate with a reasonable benefit/risk ratio.

The term: “Staphylococcus aureus bacteremia” means a S. aureus bloodstream infection.

The term “treatment,” as used herein in the context of treating an infection in a patient pertains generally to treatment and therapy in which some desired therapeutic effect is achieved, for example, the inhibition of the progress of the infection, and includes a reduction in the rate of progression of the infection, a halt in the rate of progress of the infection, alleviation of symptoms of the infection, amelioration of infection, and/or cure of the infection. For example, treatment can be the diminishment of one or several symptoms of the infection or complete eradication of the infection. Within the meaning of the present disclosure, the term “treat” also includes and/or reduce the risk of developing or worsening of an infection.

The term “patient” as used herein refers to a human.

Where ranges are given the end points of the range are included within the range.

Ceftobiprole has the structure shown below:

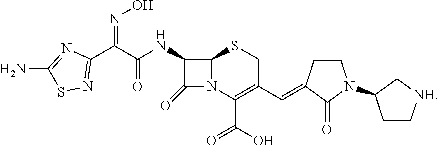 [SMILES: Nc1nc(/C(=N/O)C(=O)N[C@@H]2C(=O)N3C(C(=O)O)=C(/C=C4\CCN([C@@H]5CCNC5)C4=O)CS[C@H]23)ns1]

Ceffobiprole is administered in the form of the prodrug ceftobiprole medocaril, which has the structure shown below:

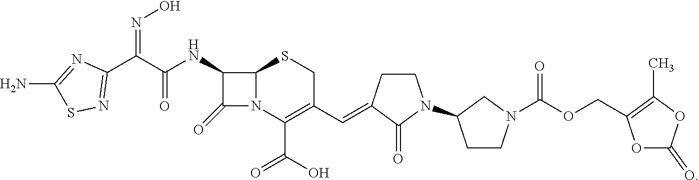 [SMILES: Cc1oc(=O)oc1COC(=O)N1CC[C@@H](N2CC/C(=C\C3=C(C(=O)O)N4C(=O)[C@@H](NC(=O)/C(=N\O)c5nsc(N)n5)[C@H]4SC3)C2=O)C1]

Ceftobiprole medocaril has the chemical name:

(6R,7R)-7-[[(2%)-2-(5-amino-1,2,4- thiadiazo)-3-yl)-2-(hydroxyimino)acetyl]amino]-3-[(E)-((3′R)-1′-[[(5-methyl-2-oxo-1,3-dioxol-4-yl)methoxy|carbonyl]-2-oxo[1,3′-bipyrrolidin]-3-ylidene]methyl]-8-oxo-5-thi a-1-azabicyclo[4.2.0]oct-2-ene-2-carboxylic acid.

The present invention provides methods of treating a S. aureus bloodstream infection (bacteremia) in a patient in need thereof, the method comprising administering ceftobiprole as ceftobiprole medocaril to the patient at a dosage corresponding to 500 mg of ceftobiprole every 6 hours on days 1 to 8 and 500 mg every 8 hours on days 9 onwards.

Correspondingly the invention provides ceftobiprole as ceftobiprole medocaril for use in a method of treating a S. aureus bloodstream infection (bacteremia) in a patient in need thereof, the method comprising administering ceftobiprole medocaril to the patient at a dosage corresponding to 500 mg of ceftobiprole every 6 hours on days 1 to 8 and 500 mg every 8 hours on days 9 onwards.

Likewise, the invention provides use of ceftobiprole as ceftobiprole medocaril in the manufacture of a medicament for treating a S. aureus bloodstream infection (bacteremia) in a patient in need thereof, the method comprising administering ceftobiprole as ceftobiprole medocaril to the patient at a dosage corresponding to 500 mg of ceftobiproie every 6 hours on days 1 to 8 and 500 mg every 8 hours on days 9 onwards,

The reference to a dosage corresponding to 500 mg of ceftobiprole refers to dose corresponding to 500 mg of the free base of ceftobiprole. The amount of ceftobiprole medocaril as the monosodium salt corresponding to 500 mg of ceftobiprole free base is 666.6 mg.

Ceftobiprole as cefobiprole medocaril is administered to the patient at a dosage corresponding to 500 mg of ceftobiprole every 6 hours on days 1 to 8. Day 1 is the first day on which the patient receives ceftobiprole. Day 2 and each subsequent administration day starts at midnight. On day 9 the patient receives the first dose of ceftobiprole 8 hours after the last dose on day 8.

The dosage regimen referred to above is used for treating adult patients, i.e. patients who are at least 18 years of age. When the patient is a pediatric patient (less than 18 years of age) the dosage of ceftobiprole may be modified as shown in Table A.

TABLE A
Dose modifications for pediatric patients

| | Body | | | |
|---|---|---|---|---|
| Age Group | Weight | | Dosing Regimen | |
| Adolescents 12 to < 18 years | ≥50 | kg | 500 | mg every 8 hours |
| | <50 | kg | 10 | mg/kg every 8 hours |
| Infants ≥ 3 months and | ≥33 | kg | 500 | mg every 8 hours |
| children < 12 years | <33 | kg | 15 | mg/kg every 8 hours |
| Term neonates and infants < | ≥4 | kg | 15 | mg/kg every 12 hours |
| 3 months | <4 | kg | 10 | mg/kg every 12 hours |

The amounts referred to in Table A correspond to ceftobiprole as free base. The amount of ceftobiprole medocaril will be correspondingly higher.

When the patient is a patient with renal impairment the dosage of ceftobiprole is usually modified as shown in Table B.

TABLE B
Dose modification for renally impaired patients

| Creatinine clearance, | |
|---|---|
| CL_{CR} (mL/min) | Dosage regimen |
| 30 to < 50 (moderate renal | 500 mg every 8 hours on days 1 to 8 |
| impairment) | 500 mg every 12 hours on days 9 onwards |
| 10 to < 30 (severe renal | 250 mg every 8 hours on days 1 to 8 |
| impairment) | 250 mg every 12 hours on days 9 onwards |
| End-stage renal disease | 250 mg every 24 hours |
| (ESRD), including | |
| hemodialysis* | |

*It is recommended that ceftobiporle is administered after hemodialysis on hemodialysis days, because ceftobiprole medocaril sodium is hemodialysable

The estimation of creatinine clearance should be based on the Cockcroft-Gault formula using actual body weight in adult patients and the Schwartz formula in paediatric patients.

The amounts referred to in Table B likewise correspond to ceftobiprole as free base. The amount of ceftobiprole medocaril will be correspondingly higher.

A course of treatment with ceftobiprole usually continues for a minimum of 21 days. In some embodiments the treatment continues for a minimum of 28 days. In some embodiments the treatment is up to 42 days. In some embodiments the treatment is a minimum of 21 days and up to 42 days. In some embodiments the treatment is a minimum of 28 days and up to 42 days. In some embodiments the treatment may continue for more than 42 days, e.g., on the basis of an individualized treatment decision from a physician.

In some embodiments the treatment is employed in order to increase the likelihood of and/or in order to achieve microbiological eradication of the infection. In some embodiments the treatment results in microbiological eradication of the infection.

In some embodiments the treatment with ceftobiprole may be employed in order to reduce the likelihood of and/or to avoid any new S. aureus complication, such as infective endocarditis, roetastatic infection and/or abscess formation. In some embodiments the treatment with ceftobiprole avoids any new S. aureus complications.

In some embodiments the patient has right-sided infective endocarditis caused by S. aureus. Infective endocarditis is an infection of the endocardium. Right-sided infective endocarditis can be classified into three epidemiological groups: intravenous drug users, intravascular device carriers, and the ‘three noes’ group (no left-sided, no device, no intravenous drug users). The modified Duke criteria (see below) is generally used for the diagnosis (San Roman et al.). In some embodiments the patient has native-valve right-sided infective endocarditis (e.g., definite native-valve right-sided infective endocarditis as determined by Modified Duke's Criteria, see below).

According to Duke's Criteria, diagnosis of infective endocarditis can be definite, possible, or rejected. A diagnosis of infective endocarditis is definite if either the following pathological or clinical criteria are met: 1. Pathologie criteria: pathologie lesions (vegetation or intracardiac abscess demonstrating active endocarditis on histology) or microorganismi: demonstrated by culture or histology of a vegetation or intracardiac abscess; 2. One of these combinations of clinical criteria (see definitions below): two major clinical criteria; one major and three minor criteria; or five minor criteria. Diagnosis of infective endocarditis is possible if one of the following combinations of clinical criteria (see definitions below) are met: one major and one minor criteria; or three minor criteria are fulfilled. Diagnosis of infective endocarditis is rejected if one of the following criteria are met: a firm alternate diagnosis is made; resolution of clinical manifestations affer ≤4 days of antibacterial treatment; no pathological evidence of infective endocarditis is found at surgery or autopsy after antibacterial treatment therapy for ≤4 days: or clinical criteria for possible or definite infective endocarditis are not met. Major criteria for the diagnosis of infective endocarditis are: 1. Positive blood culture with S. aureus from two separate blood cultures; and 2. Evidence of endocardial involvement with positive echocardiogram defined as oscillating intracardiac mass on valve or supporting structures, in the path of regurgitant jets in the absence of an alternative anatomic explanation; or abscess; or new valvular regurgitation (worsening or changing of preexisting murmuor not sufficient). Minor criteria for the diagnosis of infective endocarditis are: 1 Predisposing factor: intravenous drug use or presence of a predisposing heart condition (a valve lesion associated with significant regurgitation or turbulence of blood flow); 2. Fever ≥38° C. (100.4 ºF); 3. Vascular phenomena: major arterial emboli, septie pulmonary infarcts, mycotic aneurysm, intracranial hemorrhage, conjunctival hemorrhages or Janeway lesions; 4. Immunological phenomena: glomerulonephritis, Osler's nodes, Roth's spots, Rheumatoid factor (see Table C); and 5. A single positive blood culture with S. aureus.

TABLE C
Description of minor criteria

| Criterion | Description |
|---|---|
| Glomerulonephritis | Glomerular disease that usually presents with |
| | one of two patterns, nephrotic or nephritic, |
| | based upon the urine sediment and the degree |
| | of proteinuria. |
| Janeway lesions | Nontender erythematous macules on the palms |
| | and soles (reflecting microabscesses with |
| | neutrophil infiltration of capillaries). |
| Mycotic aneurysm | Abnormal focal arterial dilation due to S. aureus |
| | infection. |
| Osler nodes | Tender subcutaneous violaceous nodules mostly |
| | on the pads of the fingers and toes, which may |
| | also occur on the thenar and hypothenar eminences |
| | (assumed to reflect sequelae of vascular occlusion |
| | by microthrombi leading to localized |
| | immune-mediated vasculitis). |
| Rheumatoid factor | Antibodies directed against the Fe portion of |
| | immunoglobulin G (IgG); commonly measured in |
| | clinical practice, is an IgM RF. |
| Roth spots | Exudative, edematous hemorrhagic lesions of |
| | the retina with pale centers (assumed to reflect |
| | sequelae of vascular occlusion by microthrombi |
| | leading to localized immune-mediated vasculitis). |

In some embodiments the patient has a bone infection caused by S. aureus. In some embodiments the bone infection is a non-implant associated bone infection. In some embodiments the bone infection is an implant-associated bone infection. In some embodiments the bone infection is osteomyelitis. Osteomyelitis is an infection of bone resulting e.g., in its inflammatory destruction, bone necrosis, and/or new bone formation. The Waldvogel classification system describes three types of osteomyelitis: bematogenous osteomyelitis, contiguous-focus osteomyelitis (from adjacent structures such as joint spaces or soft tissues or from trauma or surgery with direct implantation of organisms), and osteomyelitis with vascular insufficiency (most commonly in patients with diabetes or peripheral vascular disease and generally involving the foot). S. aureus is the predominant cause of osteomyelitis in all of these categories. Hematogenous osteomyelitis generally involves the ends of long bones in children and adolescents and the axial skeleton in older adults, partly due to the blood supply to vertebrae in adults being more extensive than that to the long bones Diagnosis can be via positive blood culture and/or bone biopsy (Tong et al.).

In some embodiments the patient has a joint infection caused by S. aureus. In some embodiments the joint infection is a non-implant associated joint infection. In some embodiments the joint infection is an implant associated joint infection. In some embodiments the joint infection is septic arthritis, i.e. an infection in the joint (synovial) fluid and/or joint tissues. Arthrocentesis is considered to be the definitive diagnostic test for septic arthritis (Tong et al.; Alder et al.).

In some embodiments the patient has an intra-abdominal abscess caused by S. aureus. An intra-abdominal abscess is a collection of pus or infected fluid that is surrounded by inflamed tissue inside the abdomen. It can involve any abdominal organ.

In some embodiments the patient has a metastatic S. aureus infection of native tissue caused by S. aureus. Metastatic infection develops in a large number of patients with SAB, with joints and heart valves being the most commonly affected sites, but can occur almost anywhere in the body, e.g., kidney, lung, eye, brain, bone etc. (Mitchell et al.). Metastatic infections include but are not limited to: septic arthritis or bacterial joint infection/empyema; septic or suppurative thrombophlebitis; visceral soft-tissue abscesses; and septic pulmonary emboli/infarction. In some embodiments the treatment with ceftobiprole may be employed in order reduce the likelihood of and/or to avoid the formation of new S. aureus metastatic infections. In some embodiments the treatment with ceftobiprole avoids formation of any new S. aureus metastatic infections.

In some embodiments the patient has an acute bacterial skin and skin structure infection (ABSSSI) caused by S. aureus. ABSSSIs include wound infections, cellulitis/erysipelas, abscesses, and other serious skin infections.

In some embodiments the infection is caused by methicillin-resistant S. aureus. Based on the antibiotic susceptibilities, methicillin resistance in S. aureus is defined as an oxacillin minimum inhibitory concentration (MIC) of greater than or equal to 4 micrograms/ml., which is the breakpoint as defined by the Clinical & Laboratory Standards Institute. In some embodiments the infection is caused by methicillin-susceptible S. aureus, i.e. S. aureus with an oxacillin MIC of less than 4 micrograms/mL.

In some embodiments the patient has chronic intermittent dialysis (chronic intermittent dialysis and chronic intermittent hemodialysis are used herein interchangeably). Hemodialysis patients are at a greatly increased risk of SAB. The predominant risk factor for these patients is the presence of an intravascular access device and in particular the use of a cuffed, tunneled catheter (e.g., permacath) for dialysis. However, other host factors that result in an impairment of the host immune defense, including neutrophil dysfunction, iron overload, diabetes, and increased rates of colonization, may also increase the likelihood of invasive S. aureus infections (Tong et al.).

In some embodiments the patient has previously received potentially effective treatment with a different antibiotic for the same infection, e.g., the patient has persistent SAB. The different antibiotic may be selected from the group consisting of Amoxicillin/Clavulanic acid, Ampicillin/Sulbactam, Azithromycin, Cefaclor, Cefadroxil, Cefamandole, Cefalotin, Cefazolin, Cefdinir, Cefditoren pivoxil, Cefepime, Cefixime, Cefoperazone, Cefotaxime, Cefotetan, Cefoxitin, Cefpodoxime proxetil, Cefprozil, Ceflaroline fosamil, Celazidime, Ceftazidime/avibactam, Ceftibuten, Ceftizoxime, Ceftobiprole, Ceftolozane/tazobactam, Cefuroxime, Cephalexin, Ceftriaxone, Chloramphenicol, Ciprofloxacin, Clarithromycin, Clindamycin, Cloxacillin, Dalbavancin, Daptomycin, Delafloxacin, Dieloxacillin, Doripenem, Doxycycline, Ertapenem, Erythromycin, Flucloxacillin, Fosfomycin (except oral administration), Gemifloxacin, Imipenem/cilastatin, Levofloxacine, Linezolid, Lomefloxacin, Loracarbef, Meropenem, Meropenem/vaborbactam, Minocycline, Moxifloxacin, Nafcillin, Ofloxacin, Omadacycline, Oritavancin, Oxacillin, Piperacillin/tazobactam, Quinupristin/dalfopristin, Rifampin, Teicoplanin (except oral administration), Tedizolid, Telavancin, Telithromycin, Tetracycline, Ticarcillin/clavulanate, Tigecycline, Trimethoprim, Trimethoprim-sulfamethoxazole, and Vancomycin. In particular the different antibiotic is e.g., daptomycin, vancomycin or a beta-lactam antibiotic, e.g., a cephalosporin, oxacillin, nafcillin or flucloxacillin. Beta-lactam antibiotics are not usually used to treat MRSA. In some embodiments the different antibiotic is vancomycin, In some embodiments the different antibiotic is daptomycin. In some embodiments the different antibiotic is a beta-lactam antibiotic. In some embodiments the patient bas not previously received treatment with a potentially effective different antibiotic for the infection. “Potentially effective” means that the antibiotic was prescribed by a physician in order to treat the infection.

In some embodiments the treatment with ceftobiprole may be employed in order to reduce the likelihood of and/or to avoid a relapse of the infection. In some embodiments the treatment with ceftobiprole avoids a relapse of the infection.

In some embodiments the treatment with ceftobiprole may be employed in order to reduce the likelihood of and/or to avoid the infection developing resistance to the treatment. In some embodiments the treatment with ceftobiprole avoid the infection developing resistance to the treatment. In some embodiments the S. aureus ceftobiprole MIC does not increase by more than 4-fold during the treatment. In some embodiments the S. aureus ceftobiprole MIC does not increase by more than 3-fold during the treatment. In some embodiments the S. aureus ceftobiprole MIC does not increase by more than 2-fold during the treatment. In some embodiments the S. aureus ceftobiprole MIC does not increase during the treatment.

In some embodiments the S. aureus infection to be treated has a vancomycin MIC of higher than 1 mg/L. In some embodiments the S. aureus infection to be treated bas a vancomycin MIC of higher than 2 mg/L.

In some embodiments the S. aureus infection to be treated has a vancomycin MIC of higher than 3 mg/L. In some embodiments the S. aureus infection to be treated has a vancomycin MIC of higher than 4mg/L.

In some embodiments the S. aureus infection to be treated has a vancomycin MIC of 2-8 mg/L. In some embodiments the S. aureus infection to be treated has a vancomycin MIC of 4-8 mg/L. Cefobiprole and methods of preparing and formulating ceftobiprole are described in U.S. Pat No. 5,981,519, incorporated herein by reference. Ceftobiprole medocaril and methods of preparing and formulating ceftobiprole medocaril are described in U.S. Pat No. 6,232,306, incorporated herein by reference. Ceftobiprole medocaril is available commercially as the monosodium salt. Alternative phannaceutically acceptable salts may be prepared by the person skilled in the art according to their common general knowledge, e.g., as described in U.S. Pat No. 5,981,519 and U.S. Pat No. 6,232,306.

Ceftobiprole medocaril (e.g., as the monosodium salt) may be provided for use in intravenous infusion as a lyophilized power, which is reconstituted before use and then usually diluted, e.g., in sodium chloride solution for injection, dextrose solution for injection, or Lactated Ringer's solution for injection. To avoid precipitation, ceftobiprole medocaril and calcium-containing solutions, except Lactated Ringer's solution for injection, should not be mixed or administered simultaneously in the same intravenous line.

Ceftobiprole medocaril may be provided as lyophilized powder in doses (e.g., vials) containing 666.6 mg of ceftobiprole medocaril sodium salt (corresponding to 500 mg of ceftobiprole free base). Afler reconstitution in 10 mL of solution, each mL of concentrate will contain 50 mg of ceftobiprole (as 66.7 mg of ceftobiprole medocaril).

Generally ceftobiprole is administered to the patient by intravenous infusion over a period of 2 hours.

For example the lyophilized powder of ceftobiprole medocaril (e.g., the sodium salt) may be reconstituted as follows:
1. Reconstitution:
   a. For adult and pediatric patients aged ≥12 years who require an infusion solution with a ceflobiprole concentration of 2 mg/mL: with 10 ml of sterile water for injections, or with 5% (50 mg/mL) dextrose solution for injection.
   b. For pediatric patients aged <12 years who require an infusion solution with a ceftobiprole concentration of 4 mg/ml .:
      i. with 10 ml. 5% dextrose solution for injection, if further dilution with the same diluent solution (i.e., 5% dextrose solution for injection) is used, or
      ii. with 10 ml of water for injection, if further dilution with 0.9% (9 mg/mL) sodium chloride solution for injection is used
2. The vial is shaken vigorously until complete dissolution, which in some cases may take up to 10 minutes. The volume of the resulting concentrate is approximately 10.6 ml.
3. Any foam is allowed to dissipate, and the reconstituted solution should be visually inspected to ensure the product is in solution and particulate matter is absent.
4. The reconstituted concentrate contains $0 mg/mL of cefobiprole (as 66.7 mg/ml of ceftobiprole medocaril sodium) and is further diluted prior to administration.
5. It is recommended that the reconstituted solution be further dilated immediately. However, if this is not possible, the reconstituted solution may be stored at room temperature for up to 1 hour, or in a refrigerator for up to 24 hours.

The reconstituted ceftobiprole may be diluted for use in adult and pediatric patients ≥12 years as follows;
1. Preparation of 500 mg dose of ceftobiprole solution for infusion (2 mg/ml ceftobiprole)
   a. 10 ml. of the reconstituted solution is withdrawn from the vial and injected into a suitable container (e g., polyvinylchloride [PVC] or polyethylene [PE] infusion bags, glass bottles) containing 250 mL of 0.9% (9 mg/ml.) sodium chloride solution for injection, 5% dextrose solution for injection, or Lactated Ringer's solution for injection.
   b. The infusion solution is gently inverted 5-10 times to form a homogenous solution. Vigorous agitation is avoided to prevent foaming.
      i. For adult patients, the entire contents of the infusion bag is infused to administer a 500 mg dose of ceftobiprole .
      ii. For pediatric patients ≥12 years, the volume to be administered is equivalent to the calculated dose in mg/kg but does not exceed a maximum of 500 mg of ceftobiprole.
2. Preparation of 250 mg dose of ceftobiprole solution for infusion for adult patients with severe renal impairment:
   a. 5 mL of the reconstituted solution is withdrawn from the vial and injected into a suitable container (e.g., PVC or PE infusion bags, glass bottles) containing 125 ml of 0.9% (9 mg/mL) sodium chloride solution for injection, 5% (50 mg/ml) dextrose solution for injection, or Lactated Ringer's solution for injection.
   b. The infusion solution is gently inverted 5-10 times to form a homogenous solution.
   c. Vigorous agitation is avoided to prevent foaming. The entire contents of the infusion bag is infused to administer a 250 mg dose of ceftobiprole .

The reconstituted ceftobiprole may be diluted for use in pediatric patients <12 years as follows:
1. Preparation of ceftobiprole solution for infusion at a concentration of 4 mg/ml of ceffobiprole
   a. For administration via infusion bags, bottles or syringes, the reconstituted solution prepared with 10 mL. 5% dextrose solution for injection is diluted with the same diluent solution (i.e., 5% dextrose solution for injection).
      i. The reconstituted solution prepared with 10 mL water for injection solution is diluted with 0.9% sodium chloride solution for injection.
      b. 10 mL is withdrawn from an infusion container (e.g., PVC or PE infusion bags, glass bottles) containing 125 mL of diluent solution and replaced with 10 mL of the reconstituted solution withdrawn from the vial.
      c. The infusion solution is gently inverted 5-10 times to form a homogenous solution. Vigorous agitation is avoided to prevent foaming.
      d. The volume to be administered is equivalent to the calculated dose in mg/kg, but does not exceed a maximum of 500 mg of ceftobiprole.
2. For administration via a 50 mL syringe if the calculated dose does not exceed 200 mg, 4 mL of the reconstituted solution (equivalent to 200 mg ceftobiprole) prepared with 5% dextrose solution for injection or water for injection is withdrawn from the vial and diluted with 46 mL of the appropriate infusion solution diluent.
   a. The infusion solution is gently inverted 5-10 times to form a homogenous solution.
   b. Vigorous agitation is avoided to prevent foaming.
   c. The volume to be administered is equivalent to the calculated dose in mg/kg, but does not exceed a maximum of 500 mg of ceftobiprole.

Generally the reconstituted and infusion solutions is stored in a refrigerator (2° C.-8° C.) and protected from light. From a microbiological point of view, unless the method of reconstitution/dilution precludes the risk of microbiological contamination, the product should be used immediately. The infusion solution should be equilibrated to room temperature before administration and does not need to be protected from light during administration. The treatment may be for a mimumom of 21 days and/or may be for up to 42 days.

In some embodiments the invention provides a method of treating a S. aureus bloodstream infection (bacteremia) (e.g., complicated SAB) to a patient in need thereof, the method comprising administering ceftobiprole as cefiohiprole medocaril to the patient at a dosage corresponding to 500 mg of ceftobiprole every 6 hours on days 1 to 8 and 500 mg every 8 hours on days 9 onwards, wherein the patient has an infection caused by S. aureus selected from the group consisting of right-sided infective endocarditis (e.g., definite right-sided infective endocarditis), a non-implant associated bone infection (e.g. osteomyelitis) and a non-implant associated joint infection (e.g., septic arthritis). The treatment may be for a mimimum of 21 days and/or may be for up to 42 days.

In some embodiments the invention provides a method of treating a S. aureus bloodstream infection (bacteremia) (e.g., complicated SAB) to a patient in need thereof, the method comprising administering ceftobiprole as ceftobiprole medocaril to the patient at a dosage corresponding to 500 mg of ceftobiprole every 6 hours on days 1 to 8 and 500 mg every 8 hours on days 9 onwards, wherein the patient has an infection caused by S. aureus selected from the group consisting of right-sided infective endocarditis (e.g., definite right-sided infective endocarditis), a non-implant associated bone infection (e.g. osteomyelitis) and a non-implant associated joint infection (e.g., septic arthritis), and wherein the infection is caused by methicillin-susceptible S. aureus or by methicillin-resistant S. aureus. The treatment may be for a mimimum of 21 days and/or may be for up to 42 days.

In some embodiments the invention provides a method of treating a S. aureus bloodstream infection (bacteremia) (e.g., complicated SAB) to a patient in need thereof, the method comprising administering ceftobiprole as ceftobiprole medocaril to the patient at a dosage corresponding to $00 mg of ceftobiprole every 6 hours on days 1 to 8 and 500 mg every 8 hours on days 9 onwards, wherein the patient has an infection caused by S. aureus selected from the group consisting of right-sided infective endocarditis (e.g., definite right-sided infective endocarditis), a non-implant associated bone infection (e.g. osteomyelitis) and a non-implant associated joint infection (e.g., septic arthritis), and wherein the infection is caused by methicillin-susceptible S. aureus or by methicillin-resistant S. aureus, and wherein when the patient is a patient with renal impairment the dosage of ceftobiprole is modified as indicated io Table B. The treatment may be for a minimum of 21 days and/or may be for up to 42 days,

In some embodiments the invention provides a method of treating a S. aureus bloodstream infection (bacteremia) (e.g., complicated SAB) to a patient in need thereof, the method comprising administering ceftobiprole as ceftobiprole medocaril to the patient at a dosage corresponding to 500 mg of ceftobiprole every 6 hours on days 1 to & and 500 mg every 8 hours on days 9 onwards, wherein the patient bas right-sided infective endocarditis (e.g., definite right-sided infective endocarditis) caused by S. aureus. The treatment may be for a mimimum of 21 days and/or may be for up to 42 days

In some embodiments the invention provides a method of treating a S. aureus bloodstream infection (bacteremia) (e.g., complicated SAB) to a patient in need thereof, the method comprising administering ceftobiprole as ceffobiprole medocaril to the patient at a dosage corresponding to 500 mg of ceftobiprole every 6 hours on days 1 to 8 and 500 mg every & hours on days 9 onwards, wherein the patient has right-sided infective endocarditis (e.g., definite right-sided infective endocarditis) caused by S. aureus and wherein the infective endocarditis is caused by methicillin-susceptible S. aureus or by methicillin-resistant S. aureus. The treatment may be for a mimimum of 21 days and/or may be for up to 42 days.

In some embodiments the invention provides a method of treating a S. aureus bloodstream infection (bacteremia) (e.g., complicated SAB) to a patient in need thereof, the method comprising administering ceftobiprole as ceftobiprole medocaril to the patient at a dosage corresponding to 500 mg of ceftobiprole every 6 hours on days 1 to 8 and 500 mg every 8 hours on days 9 onwards, wherein the patient bas right-sided infective endocarditis (e.g., definite right-sided infective endocarditis) caused by S. aureus and wherein the infective endocarditis is caused by methicillin-susceptible S. aureus or by methicillin-resistant S. aureus, and wherein when the patient is a patient with renal impairment the dosage of ceftobiprole is modified as indicated in Table B. The treatment may be for a minimum of 21 days and/or may be for up to 42 days.

In some embodiments the invention provides a method of treating a S. aureus bloodstream infection (bacteremia) (e.g., complicated SAB) to a patient in need thereof, the method comprising administering ceftobiprole as ceftobiprole medocaril to the patient at a dosage corresponding to 500 mg of ceftobiprole every 6 hours on days 1 to & and 500 mg every 8 hours on days 9 onwards, wherein the patient has a non-1S implant associated bone infection (e.g. osteomyelitis) caused by S. aureus. The treatment may be for a mimimum of 21 days and/or may be for up to 42 days.

In some embodiments the invention provides a method of treating a S. aureus bloodstream infection (bacteremia) (c.g., complicated SAB) to a patient in need thereof, the method comprising administering ceftobiprole as ceflobiprole medocaril to the patient at a dosage corresponding to 500 mg of ceftobiprole every 6 hours on days 1 to 8 and 500 mg every 8 hours on days 9 onwards, wherein the patient has a non-implant associated bone infection (e.g. osteomyelitis) caused by S. aureus and wherein the infection is caused by methicillin-susceptible S. aureus or by methicillin-resistant S. aureus, The treatment may be for a mimimum of 21 days and/or may be for up to 42 days.

In some embodiments the invention provides a method of treating a S. aureus bloodstream infection (bacteremia) (e.g., complicated SAB) to a patient in need thereof, the method comprising administering ceftobiprole as ceftobiprole medocaril to the patient at a dosage corresponding to 500 mg of ceftobiprole every 6 hours on days 1 to 8 and 500 nig every 8 hours on days 9 onwards, wherein the patient bas a non-implant associated bone infection (e.g. osteomyelitis) caused by S. aureus and wherein the infection is caused by methicillin-susceptible S. aureus or by methicillin-resistant S. aureus, and wherein when the patient is a patient with renal impairment the dosage of ceftobiprole is modified as indicated in Table B. The treatment may be for a mimimum of 21 days and/or may be for up to 42 days.

In some embodiments the invention provides a method of treating a S. aureus bloodstream infection (bacteremia) (e.g., complicated SAB) to a patient in need thereof, the method comprising administering ceftobiprole as ceftobiprole medocaril to the patient at a dosage corresponding to 500 mg of ceftobiprole every 6 hours on days 1 to 8 and 500 mg every 8 hours on days 9 onwards, wherein the patient has a non-implant associated joint infection (e.g., septic arthritis) caused by S. aureus. The treatment may be for a mimimum of 21 days and/or may be for up to 42 days.

In some embodiments the invention provides a method of treating a S. aureus bloodstream infection (bacteremia) (e.g., complicated SAB) to a patient in need thereof, the method comprising administering ceftobiprole as ceflobiprole medocaril to the patient at a dosage corresponding to 500 mg of ceftobiprole every 6 hours on days 1 to & and 500 mg every 8 hours on days 9 onwards, wherein the patient has a non-implant associated joint infection (e.g., septic arthritis) caused by S. aureus, and wherein the infection is caused by methicillin-susceptible S. aureus or by methicillin-resistant S. aureus. The treatment may be for a mimimum of 21 days and/or may be for up to 42 days.

In some embodiments the invention provides a method of treating a S. aureus bloodstream infection (bacteremia) (e.g., complicated SAB) to a patient in need thereof, the method comprising administering cefiobiprole as ceftobiprole medocaril to the patient at a dosage corresponding to 500 mg of ceftobiprole every 6 hours on days 1 to 8 and 500 mg every 8 hours on days 9 onwards, wherein the patient has a non-implant associated joint infection (e.g., septic arthritis) caused by S. aureus and wherein the infection is caused by methicillin-susceptible S. aureus or by methicillin-resistant S. aureus, and wherein when the patient is a patient with renal impairment the dosage of ceftobiprole is modified as indicated in Table B. The treatment may be for a mimimum of 21 days and/or may be for up to 42 days.

All embodiments of the invention described herein may be combined in any combination where possible.

A number of publications are cited herein in order to more fully describe and disclose the invention and the state of the art to which the invention pertains. Each of these references is incorporated herein by reference in its entirety into the present disclosure, to the same extent as if each individual reference was specifically and individually indicated to be incorporated by reference.

Particular embodiments of the invention are described in the following Examples, which serve to illustrate the invention in more detail and should not be construed as limiting the invention in any way.

### EXAMPLES

A Phase Three study has been performed comparing ceftobiprole to daptomycin with or without optional aztreonam in the treatment of complicated SAB.

### Analysis Populations

The following analysis populations were defined for this study:

Intent-to-treat population (ITT): The ITT population consisted of all randomized patients. Patients were analyzed according to the study medication assigned at randomization.

Modified intent-to-treat population (mITT): The mITT population consisted of the subset of patients in the intention to treat population who had received any amount/dose of study medication, and who had a blood culture positive for S. aureus at baseline based on a central microbiology laboratory assessment. Patients who were missing a central microbiological assessment could be included in the mITT population if there was documented uncquivocal evidence of a baseline blood culture positive for S. aureus at the local laboratory.

Clinically evaluable population (CE): The CE population consisted of the subset of patients in the mITT population who had complied with important aspects of the study, e.g., no major protocol violations, with a completed primary outcome assessment.

Safety population: The safety population consisted of all randomized patients who received any amount/dose of study medication. Patients in the safety population were analyzed according to the first medication actually received.

Pharmacokinetic population (PK): All patients who received at least one dose of ceftobiprole and had at least one plasma-concentration measurement obtained by the appropriate methodology.

### Objectives

### Primary Objective

The primary objective of the study was to demonstrate the non-inferiority of ceftobiprole to daptomycin for overall success as assessed by an independent Data Review Committee (DRC) in the treatment of S. aureus bacteremia (SAB), including infective endocarditis (IE), at the post-treatment evaluation (PTE) visit in the modified intent-to-treat (mITT) population.

### Secondary Objectives

Secondary objectives were:
To compare ceftobiprole with daptomycin with respect to:
   1. All-cause mortality (ACM) through Day 70 (PTE visit) and Day 28 in the Intent-to-Treat (ITT) and mITT populations.
   2. Microbiological eradication rates (negative blood culture for S. aureus) at Day 4, Day 8, and the end-of-treatment (BOT) and PTE visits in the mITT and clinically evaluable (CE) populations.
   3. Overall success rates in the ITT, mITT, and CE populations:
      a) at the BOT and PTB (ITT and CE populations only) visits,
      b) at the EOT and PTE visits, for IE vs non IE SAB,
      c) at the ROT and PTE visits, by renal-function status.
   4. Development of new metastatic foci, or other complications of SAB, after Day 7 in the mITT and CE populations.
   5. Time to S. aureus bloodstream clearance in the mITT and CE populations.
   6. Safety and tolerability (Safety population).
To assess the pharmacokinetics (PK) of ceftobiprole (PK population).

### Study Design

This was a randomized, double-blind, double-dummy, active-controlled, parallel-group, multi-center study in adult hospitalized patients with SAB, including IE, which was conducted in two parts.

Part 1: As animal studies bad indicated the potential for an increased risk of convulsions with prolonged (>4 weeks) ceftobiprole treatment, Part 1 of the study enrolled an initial cohort of patients (Cohort 1) for a maximum treatment duration of 28 days (N=194 patients [49.7%]), subject to a decision to proceed earlier to Part 2.

Part 2: Following a protocol pre-defined Data and Safety Monitoring Board (DSMB) interim safety assessment, a decision was made to extend the maximum treatment duration to 42 days, and the protocol was submitted to the U.S. Food and Drug Administration (FDA) for special protocol assessment, and amended accordingly on 26 Feb. 2020. Part 2 permitted the inclusion of patients with more difficult-to-treat infections, including those with complications such as osteomyelitis and epidural or cerebral abscess, which were excluded from Cohort 1. In addition, patients with left-sided infective endocarditis (LIE) diagnosed after randomization were permitted to continue in the study.

Patients were randomized 1:1 to ceftobiprole or the comparator regimen. Randomization was stratified by study site, dialysis status, and prior antibacterial treatment use within 7 days of randomization. The three phases of the study were:
1. Screening assessments of up to 72 hours prior to randomization (with the possibility of utilizing existing transthoracic [TTE] and transesophageal [TEB] echocardiography assessments that demonstrated definite right sided infective endocarditis (RIE) within 10 days of randomization).
2. Randomization and subsequent active treatment with intravenous (i.v.) study drug (ceftobiprole or daptomycin +aztreonam).
3. Post-treatment, comprising an end-of-treatment (BOT) visit (within 72 hours of last study-drug administration), Day 35 (±3 days), Day 42 (±3 days), and a PTE visit on Day 70 (±5 days) post-randomization.
   Note: Day 35 and Day 42 could have been active treatment visits for patients in Part 2 if treatment for longer than 28 days was required. Day 35 and Day 42 were post-treatment visits for all patients in Part 1, and for patients in Part 2 who were treated for longer than 28 days but bad completed treatment by the Day 35 or Day 42 visits.

The total duration of the study for each patient was approximately 10 weeks.

### Number of Patients

390 patients were planned to be randomized, and a total of 390 patients were actually randomized and comprised the ITT population (ceftobiprole n=192; daptomycin ±aztreonam n=198). Three of these patients were excluded from the mITT population: one patient in the ceffobiprole group who did not receive study treatment, and two patients in the ceftobiprole group who did not have a positive blood culture for S aureus at baseline.

### Main Criteria for Inclusion

The study enrolled adult patients with complicated SAB, defined in this context as patients with at least one positive blood culture for S. aureus at baseline, signs and symptoms of bacteremia (fever, white blood cell (WBC) count, tachycardia, and/or hypotension), and one of the following underlying conditions or complications: chronic intermittent hemodialysis or peritoneal dialysis, persistent SAB, definitive native-valve right-sided infective endocarditis, acute bacterial skin and skin structure infections or metastatic infections of native tissue (e.g., septic arthritis, septic/suppurative thrombophlebitis, visceral abscess, septic pulmonary emboli). Patients with osteomyelitis or cerebral/epidural abscess were to be enrolled in

Part 2 only. Patients who received treatment with potentially effective antistaphylococcal systemic antibacterial treatment for more than 48 hours within 7 days before randomization were excluded from the study unless they met the criteria for persistent SAB. In this context Persistent SAB meant documented failure of bloodstream clearance, defined as a positive blood culture for S. aureus within the 72 h prior to randomization, after prior appropriate anti-staphylococcal treatment (except failure under daptomycin therapy) of at least 3 complete days.

### Administration

Ceftohiprole 500 mg (as 667 mg ceftobiprole medocaril) was administered as a 2-hour intravenous (i.v.) infusion every 6 hours in the first 8 days, and every 8 hours from Day 9 onwards (with dose and/or schedule adjusted in patients with renal impairment). Daptomycin weight-based at 6 mg/kg (up to 10 mg/kg in accordance with institutional standards) administered as 0.5-hour i.v. infusion every 24 hours (with schedule adjusted in patients with renal impairment). Optional aztreonam 1000 mg fixed dose administered as 0.5-hour i.v. infusion every 24 hours (with dose adjusted in patients with renal impairment). The target treatment duration in Part I was 21-28 days of study medication, with a maximum treatment duration of 28 days. The maximum treatment duration In Part 2 was 42 days.

### Independent Data Review Committee

An independent blinded Data Review Committee (DRC), composed of six infectious diseases experts reviewed detailed patient profiles, including imaging and microbiological and laboratory results, of all patients in the ITT population, to assess the baseline categories of complicated SAB, the response outcome for the primary endpoint, and the secondary endpoints of microbiological outcomes and death attributed to SAB. In the Statistical Analysis Plan (SAP) it was pre-specified that the baseline categories of complicated SAB were to be derived based on the study investigator assessments, except for the category of persistent SAB, which was to be based on the DRC assessment.

### Endpoints

### Primary Endpoint

The primary endpoint, assessed in the mITT population, was overall success at the PTE visit at 70 days post-randomization, based on the patient meeting all of the following criteria:
1. Patient alive at Day 70 (±5 days) post-randomization.
2. No new metastatic foci or complications of the SAB infection.
3. Resolution or improvement of SAB-related clinical signs and symptoms.
4. Two negative blood cultures for S. aureus (without any subsequent positive blood culture for S. aureus):
   at least one while the patient was on active study treatment; AND
   confirmed by at least one subsequent negative blood culture for S. aureus
      either in the period between 7 days after the EOT visit and the PTE visit
      or at the PTE visit.

Treatment failure was defined as any of the following:
1. Premature discontinuation of study treatment due to DRC-assessed lack of efficacy or for adverse events (AEs) that represented manifestation of disease progression or relapse, at any time between first dose of study drug and the PTE visit.
2. Development of new metastatic or other complications related to SAB between Day 8 and the PTE visit.
3. SAB relapse/reinfection based on evidence from a blood culture positive for S. aureus (after documented clearance of S. aureus from the bloodstream and clinical improvement) between the EOT and PTE visits.
4. Receipt of systemic non-study antibacterial treatment, other than those permitted under the protocol, for the treatment of SAB. This included patients who were prematurely discontinued from study therapy due to an AE, but who required continuation of antibacterial treatment for SAB.
5. Treatment of infections other than SAB with systemic non-study antibacterial treatment which was potentially effective against S. aureus, and which was considered by the DRC to have a relevant impact on the primary endpoint in accordance with guidelines provided in the protocol.
6. Death for any reason between first administration of study drug and the PTE visit.
7. Indeterminate outcome, defined as any data needed to determine whether the outcome was success or failure missing at the PTE visit, including but not limited to:
   a) missing PTE visit, or missing key data to evaluate the primary endpoint
   b) lost-to-follow-up, or patients who withdrew consent prior to the PTE visit
   c) patients not meeting the criteria for Success or Failure, or patients not meeting all criteria for overall success
8. Requirement for systemic antibacterial treatment for SAB beyond EOT.

### Secondary Endpoints

1. All-cause mortality assessed at Day 70 (PTE visit) in the mITT population.
2. Microbiological cradication assessed by the DRC at Day 70 (PTE visit) in the mITT population.
3. The overall success rate in the ITT, mITT, and CE populations;
   a) at the BOT and PTE (ITT and CE populations only) visits
   b) at the EOT and PTE visits, for RIE vs non-IE SAB
   c) at the EOT and PTE visits, by renal-function status
4. Development of new metastatic foci or other complications of SAB after Day 7 assessed in the mITT and CE populations.
5. Time to S. aureus bloodstream clearance, defined as the elapsed time (days) from randomization to bloodstream clearance, with bloodstream clearance defined as the occurrence of two consecutive study days with blood-culture-negative assessments for S. aureus.
6. Safety and tolerability based on incidence, type, severity, and relationship to study medication of AEs; and changes in laboratory tests
7. Pharmacokinetics of ceftobiprole in the PK population

### Statistical Methods

### Primary Efficacy Analysis

The study was designed to determine whether ceftobiprole was non-inferior to daptomycin for the outcome measure of overall success at the post-treatment evaluation (PTE) visit at Day 70 (+5 days) after randomization, in the mITT population.

The observed difference in the percentage of responders at PTE (ceftobiprole group minus the daptomycin group) was determined, and a two-sided 95% confidence interval (CI) for the observed difference was computed, with adjustment for dialysis status and prior antibacterial treatment use. Cochran-Mantel-Haenszel weights were used for the stratum weight in the calculation of the CI. The non-inferiority hypothesis test was a one-sided hypothesis test performed at the 2.5% level of significance. If the lower limit of the two-sided 95% CI for the difference in response rates in the mITT population was greater than -15%, the non-inferiority of ceftobiprole to daptomycin therapy was concluded. Sensitivity analyses of the primary endpoint were foreseen in the event of an imbalance of more than 50% between the two treatment groups in the percentage of indeterminate outcome as per DRC assessment. In addition, a sensitivity analysis was foreseen excluding patients who had COVID-19-related events that may have interfered with the evaluation of efficacy from the primary analysis.

Pre-specified analyses of the primary endpoint were performed in subgroups defined by demographic characteristics (age, gender, race, ethnicity), geographic region (Europe, North America, Latin America, Other Regions), baseline blood S. aureus (MRSA, methicillin sensitive S. aureus (MSSA) overall and by Panton-Valentine Leucocidin (PVL) status, by mecA status, oxacillin susceptibility and other genotypic markers when available, bloodstream infection type (monomicrobial, polymicrobial, bloodstream, non-bloodstream), MIC for pathogens at baseline, baseline SAB category per Investigator assessment or per

DRC assessment (primary endpoint only), known predisposing risk factors for SAB/endocarditis, type of device used (intravascular, extravascular), baseline fever status, prior antibacterial treatment (yes/no), baseline creatinine clearance categories, concomitant antibacterial treatment potentially effective against S. aureus, and by concomitant antibacterial treatment for Gram-negative coverage.

### Secondary Efficacy Analyses

The primary endpoint of overall success assessed by the DRC was also assessed at the BOT visit and in the ITT and CE populations.

ACM was assessed in the mITT and ITT populations and time to death was calculated using Kaplan-Meier (K-M) methods. Survival rates (event-free probability estimates) at study Day 28 and study Day 70 in the ITT and mITT populations were provided, and associated two-sided 95% CIs were calculated using the Greenwood formula. The hazard ratio (HR) for ACM and associated 95% CI were provided using a Cox proportional-hazard model, with treatment and actual stratumn (dialysis status and prior antibacterial treatment use) as covariates.

The observed difference in the percentage of microbiological eradication as determined by the DRC in the mITT population, and a two-sided 95% CI for the observed difference, were computed, with adjustment for actual strata (dialysis status and prior antibacterial treatment use).

S. aureus clearance was assessed by K-M methods in the mITT and CE populations using the Greenwood formula to calculate Cls of K-M estimates. The median of the observed survival time, the 25^{th} and 75^{th} percentiles, and the 95% CI were provided, and an unstratified log-rank test was performed to assess the difference between treatment groups. The hazard ratio for S. aureus clearance and associated 95% CI were also provided using a Cox proportional-hazard model, with treatment and actual stratum (dialysis status and prior antibacterial treatment nse) as covariates.

The numbers and percentages of patients who develop new metastatic foci or other complications of SAB after Day 7 were determined in each treatment group in the mITT and CE populations. The observed difference in the percentage of development of new metastatic foci or other complications of SAB after

Day 7 was determined, and a two-sided 95% CI for the observed difference computed, with adjustment for actual stratum (dialysis status and prior antibacterial treatment use).

### Other Efficacy Analyses

Mortality due to SAB as assessed by the DRC at Day 28 and Day 70 (PTE visit) (mITT and ITT populations) was analyzed as described for the secondary analysis. Investigator-assessed overall success evaluated at the BOT and PTE visits (mITT and CE populations) was analyzed as described for the primary analysis.

To study the concordance between DRC-assessed and Investigator-assessed overall success, the observed agreement relative to cach rating category individually was computed for positive agreement and negative agreement.

Bloodstream clearance of any pathogen in the mITT and CE populations was analyzed using the K-M method.

Distribution of patients at baseline and post-baseline with blood S. aureus, other blood pathogens, and non-blood pathogens, was provided for the mITT and CE populations, overall and by geographical region, as well as the distribution of blood S. aureus by virulence markers (PVL status, mecA status, oxacillin susceptibility, and other genotypic markers).

The distribution of ceftobiprole, daptomycin, and aztreonam MICs, including MIC50, MIC90, and MIC range, for blood S. aureus, MRSA, and MSSA, was tabulated for the mITT and CE populations. The shifts in ceftobiprole, daptomycin, and aztreonam MIC results from baseline to PTE, and the shifts in ceftobiprole, daptomycin, and aztreonam MIC results from baseline to the maximum value post-baseline by baseline infecting organisms (i.e., pathogens), were presented overall and by geographical region in the mITT and CE populations.

Information on health economics outcome measures from baseline to the PTE visit was collected at the PTE visit, to enable analyses of length of stay in the hospital, healthcare encounters and re-hospitalizations due to AEs.

### Summary of Results and Conclusions

### Patient Disposition

The ITT population comprised 390 patients (ceftobiprole: n=192; daptomycin with or without aztreonam: n=198). One patient in the ceftobiprole group who did not receive study treatment, and two patients in the ceftobiprole group who did not have a positive blood culture for S. aureus at baseline, were excluded from the mITT population, which therefore comprised 387 patients (ceftobiprole: n=189; daptomycin±aztreonam: n=198).

Thirty-four patients in the ceftobiprole group and 29 patients in the daptomycin±aztreonam group discontinued the study in the mITT population. The main reasons were death (ceftobiprole n=17, 9.0%; daptomycin±aztreonam n=18, 9.1%) and withdrawal by subject (ceftobiprole n=10, 5.3%; daptomycin ±aztreonam n=6, 3.0%).

Forty patients in the ceftobiprole group, and 35 in the daptomycin±aztreonam group, discontinued study treatment in the mITT population. The main reasons were death (ceftobiprole a=9, 4.8%; daptomycin±aztreonam n=6, 3.0%), related AE (ceftobiprole n=9, 4.8%; daptomycin +aztroonam n=3, 1.5%), non-related AE (ceftobiprole n=6, 3.2%; daptomycin±aztreonam n=6, 3.0%), and withdrawal by subject (ceftobiprole n=6, 3.2%; daptomycin : aztreonam n=3, 1.5%).

Treatment discontinuation rates were numerically slightly higher in the ceftobiprole group than in the daptomycin±aztreonam group at Days & (9.5% vs 7.1%), 15 (15.3% vs 12.6%), 22 (19.6% vs 17.2%), 29 (20.6% vs 17.7%), 36 (20.6% vs 17.7%), and 43 (21.2% vs 17.7%).

The CE population comprised 330 patients (ceftobiprole: n=163; daptomycin±aztreonam; n=167), the Safety population comprised 389 patients (ceftobiprole: n=191; daptomycin±aztreonam: n=198), and the PK population comprised 183 ceftobiprole-treated patients.

### Demographic and Baseline Characteristics

Demographic and baseline characteristics were balanced across the two treatment groups.

The median age in the mITT population was 58 years (range 19-91). More males (69.3%) than females (30.7%) were enrolled, and most patients were classified as White (95.9%). The majority of patients were recruited in Europe (93.0%), with Ukraine as the highest enrolling country (46.$%) vs 4.4% of patients enrolled in other regions, and 2.6% in the USA.

94 patients (24.3%) had a baseline MRSA isolate (ceftobiprole n=45, daptomycin±aztreonam n=49). The most frequent categories of complicated SAB were ABSSSI (61.2%), intra-abdominal abscesses (14.2%), chronic intermittent dialysis (12.7%), and septic arthritis (10.6%). Thirty patients (7.8%) with complicated SAB had osteomyelitis (ceffobiprole n=13, daptomycin±aztreonam n=17). Twenty-five patients (6.5%) with complicated SAB had definite RIE (ceftobiprole n=15, daptomycin±aztreonam n=10).

70.5% of patients bad received potentially effective prior antibiotics within 7 days of randomization.

### Efficacy Results

### Primary Endpoint

The primary endpoint of the study was met, demonstrating the non-inferiority of ceftobiprole to daptomycin±aztreonam in terms of overall success at the PTE visit at 70 days post-randomization in the mITT population.

In the mITT population, 132 out of 189 patients (69.8%) in the ceffobiprole group were responders (95% CI: 62.8, 76.3), and 136 out of 198 patients (68.7%) patients in the daptomycin±aztreonam were responders (95% CL: 61.7, 75.1). The adjusted proportion difference (ceftobiprole minus daptomycin±aztreonam) was 2.0% (95% CI: −7.1, 11.1). Non-inferiority of ceftobiprole to daptomycin±aztreonam was demonstrated, as the lower limit of the two-sided 95% CI for the difference in response (ceftobiprole minus daptomycin±aztreonam) of −7.1% was greater than the pre-specified −15% non-inferiority margin.

3S

A sensitivity analysis excluding eight patients with COVID-19 related events that may have interfered with the evaluation of efficacy showed results consistent with the primary analysis.

The results observed in the CE population were consistent with those seen in the mITT population.

There were no statistically significant differences in the primary endpoint results in the ITT population in subgroup analyses according to gender or ethnicity, causative pathogen (MRSA, MSSA), or baseline creatinine clearance (CL_{CB}).

In the age group of 18-34 years, a higher response rate was observed in the ceftobiprole group (17/20, 85.0%) than in the daptomycin±aztreonam group (11/19, 57.9%), and in non-white patients a lower response rate was observed in the ceftobiprole group (5/10, 50.0%) compared to the daptomycin±aztreonam group (5/6, 83.3%).

Results of the primary and key secondary endpoints are summarized in Table 1.

TABLE 1
Primary and key secondary endpoint analyses

| | | |
|---|---|---|
| | | Adjusted* difference |
| | | ceftobiprole − |
| | | daptomycin ± |
| | Daptomycin ± | aztrconans |
| Ceftobiprole | aztreonam | (95% CI) |

Primary endpoint analyses

| | | | |
|---|---|---|---|
| DRC overall success at PTE (mITT), | 132/189 (69.8) | 136/198 (68.7) | 2.0% (−7.1, 11.1) |
| n/N (%) | | | |
| DRC overall success at PTE (CE), | 127/163 (77.9) | 130/167 (77.8) | 0.6% (−8.3. 9.5) |
| n/N (%) | | | |

Secondary endpoint analyses (mITT)

| | | | |
|---|---|---|---|
| ACM through PTE, n/N (%) | 17/189 (9.0) | 18/198 (9.1) | −0.5% (−6.2, 5.2) |
| Death due to SAB (DRC), n/N (%) | 7/189 (3.7) | 6/198 (3.0) | 0.4% (−3.2, 4.0) |
| Microbiological response at PTE, | 155/189 (82.0) | 153/198 (77.3) | 5.1% (−2.9, 13.0) |
| n/N (%) | | | |
| Patients with development of | 11/189 (5.8) | 11/198 (5.6) | 0.1% (−4.6, 4.8) |
| new metastatic foci or other | | | |
| complications of SAB after Day | | | |
| 7, n/N (%) | | | |
| Time (days) to S. aureus | 4 (3, 5) | 4 (3, 5) | |
| bloodstream clearance, Median (95% CI) | | | |

*Cochran-Mantel-Haenszel weights method adjusted for actual stratum (dialysis status and prior antibacterial treatment use)
DRC = Data Review Committee; PTE = Post-treatment evaluation; mITT = Modified Intent-to-treat; CE = Clinically Evaluable; SAB = Staphylococcus aureus bacteremia.

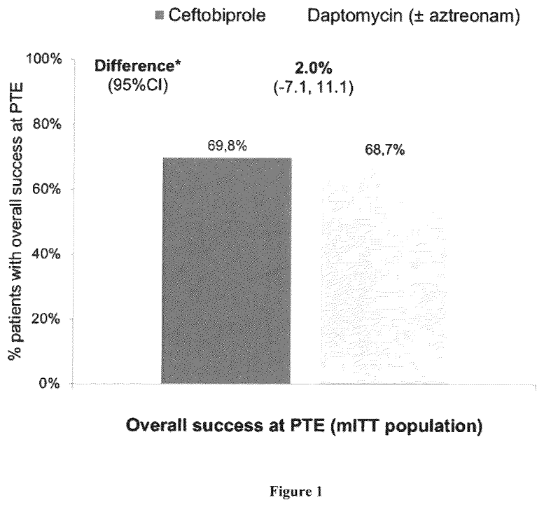

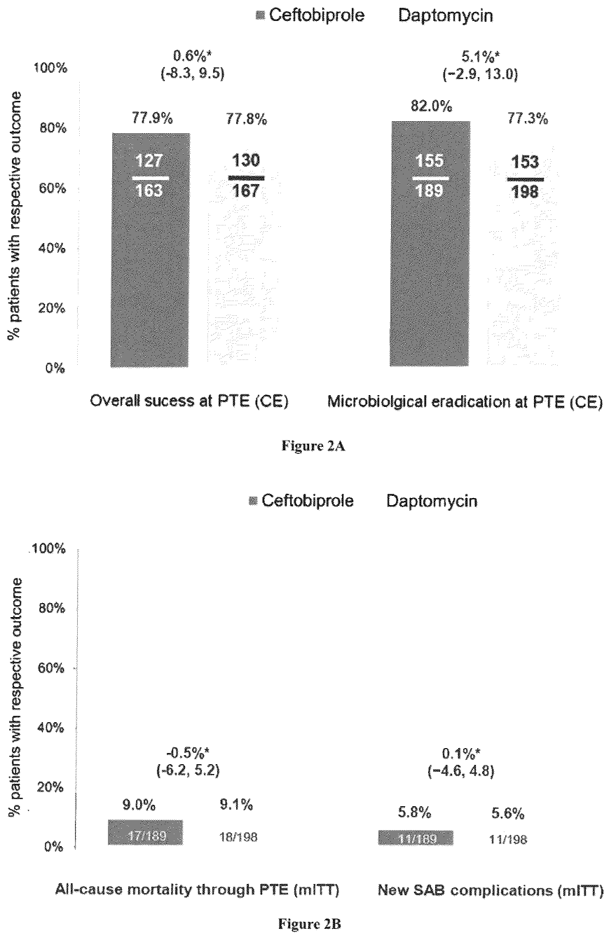

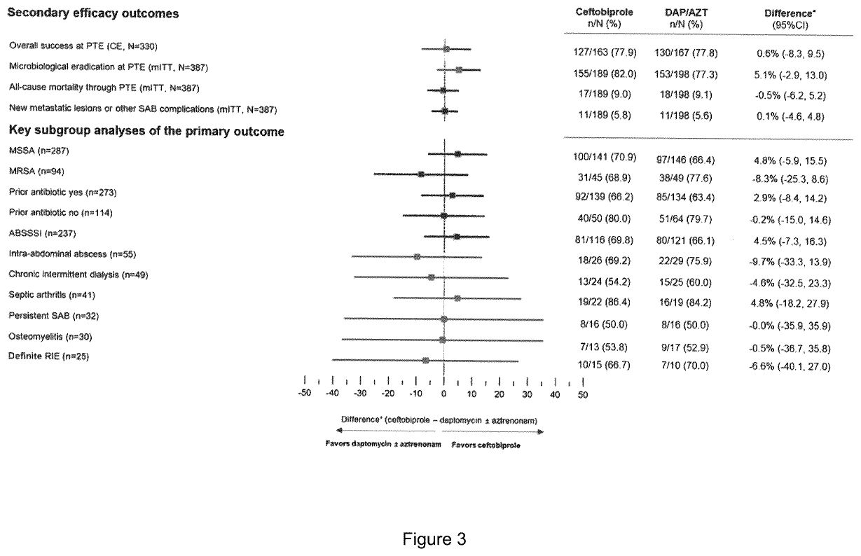

Results of the primary endpoint in MSSA and MRSA, and the secondary endpoints including in the different categories of complicated SAB, are shown in FIGS. 1-3. Results of the secondary endpoints are shown in FIG. 2.

### Main

The numbers of patients in the mITT population who died through the PTE visit were 17 in the ceftobiprole group (9.0%) and 18 in the daptomycin +aztreonam group (9.1%). The proportion difference was −0.5% (95% CI: −6.2, 5.2),

The numbers of patients in the mITT population who experienced new metastatic foci or other complications of SAB after Day 7 were 11 in the ceftobiprole group (5.8%) and 11 in the daptomycin±aztreonam group (5.6%). The proportion difference was 0.1% (95% CI: −4.6, 4.8).

The numbers of patients in the mITT population with microbiological eradication at the PTE visit were 155 in the ceftobiprole group (82.0%; 95% CI: 75.8, 87.2) and 153 in the daptomycin±aztreonam group (77.3%; 95% CI: 70.8, 82.9). The proportion difference was 5.1% (95% CI: −2.9, 13.0). The median time to S. aureus bloodstream clearance was 4 days in both treatment groups.

### Other Secondary Efficacy Endpoints

Investigator-assessed overall success evaluated at the PTE visit in the mITT population was 73.5% in the ceftobiprole group, and 69.7% in the daptomycin±aztreonam group (adjusted proportion difference 4.7%, 95% CI: −4.2, 13.5).

There were no notable overall differences between the two treatment groups in health economic endpoints.

### Other Microbiology Results

In the ceftobiprole group, MICs were determined in 141 MSSA samples and 43 MRSA samples. In the MSSA samples, the MIC range for ceftobiprole was 0.12 to 1 mg/L, with 99% of isolates at either 0.25 mg/L (51%) or 0.5 mg/L (48%). In the MRSA samples, the MIC for ceftobiprole range was 0.5 to 2 mg/L, with 14% of isolates at 0.5 mg/L, 58% at 1 mg/L, and 28% at 2 mg/L. There was no apparent correlation in the ceftobiprole group between MICs and clinical failure.

For daptomycin, MICs were determined in 145 MSSA samples and 49 MRSA samples. In the MSSA samples, the MIC range for daptomycin was 0.25 to 1 mg/L, with 99% of isolates at either 0.25 mg/L (28%) or at 0.5 mg/L (71%). In the MRSA samples, the MIC range for daptomycin was also 0.25 to 1 mg/L, with 22% of isolates at 0.25 mg/L, 76% at 0.5 mg/L, and 2% at 1 mg/l. There was no apparent correlation in the daptomycin±aztreonam group between MICs and clinical failure.

Clonal strain type analyses in MRSA isolates (ceftobiprole n=43, daptomycin±aztreonam n=47), based on multilocus sequence testing (MLST), spa typing, and SCCmec typing, identified 44 MRSA isolates (49%) with a USA or USA Latin American variant (LAV) cional strain type. Overall success at the PTE visits was observed in 16/21 patients (76.2%) with a USA clonal strain type in the ceftobiprole group, and 16/23 (69.6%) in the daptomycin±aztreonam group.

No on-treatment increases in cefobiprole MICs by ≥four-fold were observed. On-treatment increases in daptomycin MICs by ≥four-fold were observed in three patients (1.5%) in the daptomycin±aztreonam group (two MSSA, one MRSA).

### Pharmacokinetic Results

The pharmacokinetic results are shown in Table 2.

TABLE 2
Time to S. aureus bloodstream clearance

| | S. aureus (overall) | | MSSA | | MRSA | |
|---|---|---|---|---|---|---|
| Endpoint | Ceftobiprole | Daptomycin | Ceftobiprole | Daptomycin | Ceftobiprole | Daptomycin |
| (mITT) | N = 189 | N = 198 | N = 141 | N = 146 | N = 45 | N = 49 |
| Patients | 94.2% | 92.9% | 94.3% | 95.2% | 93.3% | 87.8% |
| with | | | | | | |
| clearance | | | | | | |
| achieved | | | | | | |
| Median | 4 | 4 | 3 | 4 | 5 | 5 |
| time (days) | (3, 5) | (3, 5) | (3, 5) | (3, 5) | (3, 6) | (4, 6) |
| clearance | | | | | | |
| (95% CI) | | | | | | |

Bloodstream clearance defined as two consecutive study days with blood-culture-negative assessments for S. aureus, without any subsequent S. aureus relapse or reinfection per DRC assessment. The first day with negative blood culture was used for calculating time to bloodstream clearance. Patients without clearance were censored at the last study visit.

### Safety Results

Ceftobiprole was well tolerated by patients with complicated SAB, and the safety profile of ceftobiprole in this study was consistent with the known safety profile.

Of the 389 patients in the Safety population, 238 (61.2%) experienced an AE (ceftobiprole n=121, 63.4%; daptomycin±aztreonam n=117, 59.1%), 81 patients (20.8%) experienced a serious adverse event (SAE) (ceftobiprole n=36, 18,8%; daptomycin±aztreonam n=45, 22.7%), and 67 patients experienced a severe AE (ceftobiprole n=29, 15.2%; daptomycin±aztreonam n=38, 19.2%). Adverse events leading to discontinuation were reported for 36 patients (ceftobiprole n=18, 9.4%; daptomycin±aztreonam n=18, 9.1%)

Study-drug-related AEs were more frequent in the ceftobiprole group (n=25, 13.1%) than in the daptomycin +aztreonam group (n=11, 5.6%); this was mainly driven by a higher percentage of gastrointestinal AEs, including mild/moderate cases of nausea. The frequency and spectrum of gastrointestinal side effects observed in this study was similar to the safety profile observed in Phase 3 studies with ceftobiprole in ABSSSI and CABP.

There were no reported AEs related to Clostridioides difficile in either treatment group.

Drug-related AEs leading to discontinuation were reported for 12 patients (ceftobiprole n=9 patients, 4.7%; daptomycin +aztreonam n=3 patients, 1.5%). In the ceftobiprole group, these drug-related AEs included allergy-type events (n=4), nausea/vomiting (n=4), and seizure, leukopenia, and hepatic function test abnormalities (n=1 each). In the daptomycin group these drug-related AEs included cosinophilic pneumonia (n=2) and myopathy (n=1).

The most frequently reported AEs were anemia (ceftobiprole 11.0% of patients; daptomycin±aztreonam 12.1%), nausea (ceftobiprole 10.5%; daptomycin±aztreonam 4.0%) and gamma-glutamyl-transferase increased (ceftobiprole 6.3%; daptomycin±aztreonam 7.6%).

### Relapse and Resistance Development

DRC-assessed SAB relapses were observed in 4 patients on daptomycin (3.0%, 2 MSSA, 2 MRSA) and 2 patients (1.1%, all MSSA) on ceftobiprofe.

On-treatment MIC increases of 24 fold were observed in 3 patients on daptomycin (1.5%, 2 MSSA, 1 MRSA) but not on ceffobiprole.

16 patients had high vancomycin MICs of 2-8 mg/L (8 MRSA, & MSSA); in these patients overall success rates were 87.5% (7/8) with ceftobiprole and 50.0% (4/8) with daptomycin.

### Conclusions

Ceftobiprole met the primary objective for this study, and showed similar outcomes compared to daptomycin & aztreonam in all of the secondary efficacy objectives. For the primary objective, the non-inferiority of ceftobiprole (greater than the pre-specified -15% non-inferiority margin) to daptomycin±aztreonam in the mITT population was demonstrated, meeting the primary endpoint of overall success at the PTE visit (70 days post-randomization) as assessed by the independent blinded DRC. The results for the primary and secondary outcomes were consistent in the CB population.

Ceftobiprole provided a similar benefit to daptomycin +aztreonam in patients with a representative selection of complicated SAB, including 94 patients (24.3%) with MRSA, and a broad range of underlying causes or complications of SAB, including skin sources (61.2%), intra-abdominal abscesses (14.2%), chronic intermittent dialysis (12.7%), septic arthritis (10.6%), and osteomyelitis (7.8%). Twenty-five patients (6.5%) with complicated SAB had definite RIE (ceftobiprole n=15, daptomycin±aztreonam n=10).

Ceftobiprole was found to be safe and well tolerated at the given dose levels in patients with complicated SAB, confirming its well-established safety and tolerability profile. More patients in the ceftobiprole group experienced gastrointestinal AEs than in the daptomycin +aztreonam group, a finding that is consistent with other Phase 3 studies with ceftobiprole in ABSSSI and CABP. Numerically fewer patients experienced SABs and severe AEs in the ceftobiprole group in this study.

The study demonstrates that ceftobiprole may be effective as a single-agent treatment for complicated SAB.

### REFERENCES

Tong S Y, Davis J S, Eichenberger E, Holland T L, Fowler V G Jr. Staphylococcus aureus infections: epidemiology, pathophysiology, clinical manifestations, and management. Clin. Microbiol. Rev. 28(3), 603-661 (2015).
Asgeirsson H, Thalme A, Weiland O. Staphylococcus aureus bacteraemia and endocarditis-epidemiology and outcome: a review. Infect.Dis. (Lond) 50(3), 175-192 (2018).

Bassetti M, Peghin M, Trecarichi E M et al. Characteristics of Staphylococcus aureus bacieraemia and predictors of early and late mortality. PLOS ONE 12(2), e0170236 (2017).

Austin E D, Sullivan S S, Nacesie N et al. Reduced mortality of Staphylococcus aureus bacteremia in a retrospective cohort study of 2139 patients: 2007-2015. Clin. Infect. Dis. doi: 10.1093/cid/ciz498 (2019) (Epub ahead of print)

Van Hal S J, Jensen S O, Vaska V I, Espedido B A, Paterson D L, Gosbell I B. Predictors of mortality in Staphylococcus aureus bacteremia. Clin. Microbiol. Rev. 25(2), 362-386 (2012).

Thwaites G E, Edgeworth J D, Gkrania-Klotsas E et al. Clinical management of Staphylococcus aureus bacteraemia. Lancet Infect. Dis. 11(3), 208-222 (2011).

Holland T L, Chambers H P, Boucher H W et al. Considerations for clinical trials of Staphylococcus aureus bloodstream infection in adults. Clin. Infect, Dis. 68(5), 865.872 (2019).

Fowler V G Jr, Boucher H W, Corey G R et al. Daptomycin versus standard therapy for bacteremia and endocarditis caused by Staphylococcus aureus. N. Engl. J. Med. 355(7), 653-665 (2006).

Sharma M, Riederer K, Chase P, Khatib R. High rate of decreasing daptomycin susceptibility during the treatment of persistent Staphylococcus aureus bacteremia. Bur. J. Clin. Microbiol. Infect. Dis. 27(6), 433-437 (2008).

Gasch O, Camoez M, Dominguez M A et al. Emergence of resistance to daptomycin in a cohort of patients with methicillin-resistant Staphylococcus aureus persistent bacteraemia treated with daptomycin. J. Antimicrob. Chemother. 69(2), 568-571 (2014).

Zhang S, Sun X, Chang W, Dai Y, Ma X. Systematic review and meta-analysis of the epidemiology of vancomycin-intermediate and heterogeneous vancomycin-intermediate Staphylococcus aureus isolates. PLOS ONE 10(8), e0136082 (2015).

Liapíkou A, Cilloniz C, Torres A. Ceftobiprole for the treatment of pneumonia: a European perspective. Drug Des. Devel. Ther. 9, 4565-4572 (2015).

Morosini M I, Diez-Aguilar M, Canton R. Mechanisms of action and antimicrobial activity of ceftobiprole. Rev. Esp. Quimioter. 32(Suppl. 3), 3-10 (2019).

Giacobbe D R, De Rosa FG , Del Bono V et al. Ceftobiprole: drug evaluation and place in therapy. Expert Rev. Anti. Infect. Ther. 17(9), 689-698 (2019).

Pfaller M A, Flamm R K, Mendes R E et al. Ceftobiprole activity against Gram-positive and -negative pathogens collected from the United States in 2006 and 2016. Antimicrob. Agents Chemother. 63(1), pii: e01566-e01518 (2019).

Overcash J S, Kim C, Keech R et al. Ceftobiprole Compared With Vancomycin Plus Aztreonam in the Treatment of Acute Bacterial Skin and Skin Structure Infections: Results of a Phase 3, Randomized, Double-blind Trial (TARGET). Clin Infect Dis 73(7), €1507-e1517 (2021).

San Roman J A, Lopez J Right-sided infective endocarditis, The ESC Textbook of Cardiovascular Medicine (3 edn). Ch 36.6, 1727-1728 (2018).

Alder KD, Lee I, Munger A M Intracellular Staphylococcus aureus in bone and joint infections: A mechanism of disease recurrence, inflammation, and bone and cartilage destruction. Bone, 141, 115568 (2020).

Mitchell D H, Howden B P Diagnosis and management of Staphylococcus aureus bacteraemia. Intern Med J. 35 Suppl 2, S17-24 (2005).

It is to be understood that the invention is not limited to the particular embodiments of the invention described above, as variations of the particular embodiments may be made and still fall within the scope of the appended claims.

1. A method of treating a Staphylococcus aureus bloodstream infection (bacteremia) in a patient in need thereof, the method comprising administering ceftobiprole as ceftobiprole medocaril to the patient at a dosage corresponding to of 500 mg of ceftobiprole every 6 hours on days 1 to 8 and 500 mg every 8 hours on days 9 onwards.

2. The method of claim 1, wherein ceftobiprole is administered to the patient for a minimum of 21 days.

3. The method of claim 1, wherein ceftobiprole is administered to the patient for up to 42 days.

4. The method of claim 1, wherein the S. aureus bacteremia (SAB) infection is complicated SAB. 5, The method of claim 1, wherein the treatment achieves microbiological eradication of the infection.

6. The method of claim 1, wherein the treatment avoids the formation of new S. aureus metastatic infections.

7. The method of claim 1, wherein the treatment avoids any new S. aureus complications.

8. The method of claim 1, wherein the patient bas right-sided infective endocarditis caused by S. aureus.

9. The method of claim 8, wherein the right-sided infective endocarditis is definite right-sided infective endocarditis.

10. The method of claim 8, wherein the right-sided infective endocarditis is definite native-valve right-sided infective endocarditis.

11. The method of claim 1, wherein the patient has a bone infection caused by S. aureus.

12. The method of claim 11, wherein the bone infection is a non-implant associated bone infection.

13. The method of claim 12, wherein the bone infection is osteomyelitis.

14. The method of claim 1, wherein the patient has a joint infection caused by S. aureus.

15. The method of claim 14, wherein the joint infection is a non-implant associated bone infection.

16. The method of claim 15, wherein the joint infection is septic arthritis.

17. The method of claim 1, wherein the patient has an intra-abdominal abscess caused by S. aureus.

18. The method of claim 1, wherein the patient has a metastatic infection of native tissue caused by S. aureus.

19. The method of claim 1, wherein the patient has an acute bacterial skin and skin structure infection (ABSSSI) caused by S. aureus.

20. The method of claim 1, wherein the infection is caused by methicillin-susceptible S. aureus.

21. The method of claim 1, wherein the infection is caused by methicillin-resistant S. aureus.

22. The method of claim 1, wherein the patient is a chronic intermittent dialysis patient.

23. The method of claim 1, wherein the patient has previously received potentially effective treatment with a different antibiotic for the infection.

24. The method of claim 23, wherein the different antibiotic is vancomycin.

25. The method of claim 23, wherein the different antibiotic is daptomycin.

26. The method of claim 23, wherein the different antibiotic is a beta-lactam antibiotic.

27. The method of claim 1, wherein the patient has persistent SAB.

28. The method of claim 1, wherein the treatment with ceftobiprole avoids a relapse of the infection.

29. The method of claim 1, wherein the S. aureus ceftobiprole MIC does not increase by more than 4-fold during the treatment.

30. The method of claim 1, wherein the S. aureus ceftobiprole MIC does not increase by more than 2-fold during the treatment.

31. The method of claim 1, wherein the S. aureus ceftobiprole MIC does not increase during the treatment.

32. The method of claim 1, wherein the S. aureus has a vancomycin MIC of higher than 2 mg/L.

33. The method of claim 1, wherein the S. aureus has a vancomycin MIC of higher than 4 mg/L.

34. The method of claim 1, wherein when the patient is a pediatric patient the dosage of ceftobiprole is as follows:

| | Body | | | |
|---|---|---|---|---|
| Age Group | Weight | | Dosing Regimen | |
| Adolescents 12 to < 18 | ≥50 | kg | 500 | mg every 8 hours |
| years | <50 | kg | 10 | mg/kg every 8 hours |
| Infants ≥ 3 months and | ≥33 | kg | 500 | mg every 8 hours |
| children < 12 years | <33 | kg | 15 | mg/kg every 8 hours |
| Term neonates and infants < | ≥4 | kg | 15 | mg/kg every 12 hours |
| 3 months | <4 | kg | 10 | mg/kg every 12 hours |

35. The method of claim 1, wherein when the patient is a patient with renal impairment the dosage of ceftobiprole is as follows:

| Creatinine clearance, | |
|---|---|
| CL_{CR} (mL/min) | Dosage regimen |
| 30 to < 50 (moderate renal | 500 mg every 8 hours on days 1 to 8 |
| impairment) | 500 mg every 12 hours on days 9 onwards |
| 10 to < 30 (severe renal | 250 mg every 8 hours on days 1 to 8 |
| impairment) | 250 mg every 12 hours on days 9 onwards |
| end-stage renal disease | 250 mg every 24 hours |
| (ESRD), including | |
| hemodialysis | |

36. The method of claim 1, wherein the ceftobiprole is administered as the sodium salt of ceftobiprole medocaril.

37. The method according to claim 1, wherein ceftobiprole medocaril is administered to the patient by injection.

38. The method according to claim 1, wherein ceftobiprole medocaril is administered to the patient by intravenous infusion.

39. The method of claim 1, wherein a dose of ceftobiprole is administered to the patient as a 2-hour intravenous infusion.

40. The method according to claim 1, wherein 500 mg of ceftobiprole corresponds to 666.6 mg of the prodrug ceftobiprole medocaril.

41. The method of claim 1, wherein the patient has an infection caused by S. aureus selected from the group consisting of right-sided infective endocarditis, a non-implant associated bone infection and a non-implant associated joint infection.

42. The method of claim 40, wherein the infection is caused by methicillin-susceptible S. aureus or by methicillin-resistant S. aureus, wherein the treatment is for up to 42 days and wherein when the patient is a patient with renal impairment the dosage of ceftobiprole is as follows:

| Creatinine clearance, | |
|---|---|
| CL_{CR} (mL/min) | Dosage regimen |
| 30 to < 50 (moderate renal | 500 mg every 8 hours on days 1 to 8 |
| impairment) | 500 mg every 12 hours on days 9 onwards |
| 10 to < 30 (severe renal | 250 mg every 8 hours on days 1 to 8 |
| impairment) | 250 mg every 12 hours on days 9 onwards |
| end-stage renal disease | 250 mg every 24 hours |
| (ESRD), including | |
| hemodialysis | |

43. The method of claim 42, wherein the infection is right-sided infective endocarditis.

44. The method of claim 43, wherein the infection is definite right-sided infective endocarditis.

45. The method of claim 42, wherein the infection is a non-implant associated bone infection.

46. The method of claim 45, wherein the bone infection is osteomyelitis.

47. The method of claim 42, wherein the infection is a non-implant associated joint infection.

48. The method of claim 47, wherein the joint infection is septic arthritis.

49. A pharmaceutical product comprising (i) a container containing ceftobiprole as ceftobiprole medocaril and (ii) instructions for using the ceftobiprole as defined in claim 1.

50. A method for the treatment of adult patients with Staphylococcus aureus bloodstream infection (bacteremia), including those with right-sided infective endocarditis, caused by methicillin-susceptible and methicillin-resistant isolates; the method comprising administering ceftobiprole as ceftobiprole medocaril to the patient at a dosage corresponding to of 500 mg of ceftobiprole every 6 hours on days 1 to 8 and 500 mg every 8 hours on days 9 onwards.
